(19) United States
(12) Patent Application Publication
(10) Pub. No.: US 2022/0047026 A1
(43) Pub. Date: Feb. 17, 2022

(54) Cooperating eyeliner and adhesive for applying false or artificial eyelashes to an eyelid, system, and related method

(71) Applicant: Moxielash, Inc, La Jolla, CA (US)

(72) Inventors: Ice Brennan, North Wales, PA (US); Xiwu Zhang, Shantou (CN); Mingtao Zahng, Shantou (CN)

(21) Appl. No.: 17/401,346

(22) Filed: Aug. 13, 2021

Related U.S. Application Data
(60) Provisional application No. 63/065,710, filed on Aug. 14, 2020.

(51) Int. Cl. A41G 5/02; A61K 8/06; A61K 8/81; A61K 8/34; A61K 8/37; A61K 8/19; A61K 8/73; A61K 8/49; A61Q 1/10; A61K 8/368

(52) U.S. Cl. CPC A41G 5/02; A61K 8/06; A61K 8/8176; A61K 8/345; A61K 8/375; A61K 2800/884; A61K 8/731; A61K 8/498; A61Q 1/10; A61K 8/368; A61K 2800/43; A61K 8/19

(57) ABSTRACT

Disclosed are reusable false lashes that employ the latest nano technology wherein the nano glue is already built-in on the lashes. The lash features a special layer of pressure sensitive adhesive that may suitably be applied on the back of the base of false lashes so that the lashes may be easily adhered to human skin with strength. The lash is configured to use a cooperating eyeliner that may be applied beforehand to assist in adhering the false lash to the skin. A lash eyeliner system allows a user to simply clean or rinse the lash base after use with water so that the lashes can be reused.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the field of adhesives and eyeliners that cooperate to apply false or artificial eyelashes to an eyelid.

### Background of the Invention

False or artificial eyelashes (hereinafter “false lashes”) are prosthetics for changing or supplementing the aesthetic of a user's natural eyelashes. Traditionally, false lashes are defined by animal hair (e.g., mink hair) or other hair-like fibers or strands tied to a base or strip of flexible material (e.g., silk, nylon, or other plastic material). Usually, the base or strip has a length approximating the width of a user's upper eyelid so that it can be trimmed and adhered to the eyelid (just above the user's real eyelashes) via surgical glue or other adhesive substance.

Glue-on false lashes are useful for changing or supplementing a user's natural eyelashes, however, these traditional eyelash prosthetics are not satisfactory in every instance. One unsatisfactory aspect of traditional false eyelashes is that spreading the surgical glue onto the base or strip of flexible material is tedious and can be time consuming for first-time users. Another unsatisfactory aspect of traditional false eyelashes is that the surgical glue is only good for a single use, so the glue must be replaced after every application of the false eyelash. The single use nature of the glue is particularly problematic whenever the strip is misapplied to the eyelid because the glue and the eyelash must be removed from the eyelid and the glue (and sometimes the entire false eyelash) must be replaced without the user having enjoyed the outfit. Additionally, the glue may be an irritant to users that have sensitive skin. Finally, glue-on false lashes can only be used one or two times before the lashes are ruined.

Magnetic eyelashes are one alternative to glue-on false lashes. Magnetic lashes are like any typical false lashes in that they have a base or strip with hair-like fibers attached. Unlike typical glue-on lashes, magnetic lashes have pieces of magnetic material secured to the base so that the false lashes are secured to an eyelid via magnetisms between the magnets and eyeliner makeup mixed with metallic particles. See, e.g., U.S. Pat. No. 3,516,422 (issued Jun. 23, 1970). Unfortunately, the magnets are uncomfortable and the metallic particles in the eyeliner can causes the makeup to be dry and difficult to remove after use. Also, the eyeliner can be an irritant to the eye during application or removal.

A third alternative to glue-on lashes are stick-on lashes. Stick-on lashes differ from glue-on lashes in that a pressure sensitive adhesive has been pre-applied to the false lashes. Because the adhesive has been pre-applied, a user simply has to stick the lashes onto the eyelid. Stick-on lashes are easier to apply than glue-on lashes, but the adhesive that has typically been pre-applied to stick-on lashes is typically weaker than the surgical adhesive used for glue-on lashes. As a result, stick-on lashes are know to fall off at undesirable times and are typically not reusable.

### SUMMARY OF THE INVENTION

In view of the foregoing, an object of this specification is to disclose reusable false lashes that employ the latest nano technology to accomplish a simple application. In a preferred embodiment, a special layer of pressure sensitive adhesive may suitably be applied on the back of the base of false lashes so that the lashes may be easily adhered to human skin with strength. A cooperating eyeliner can be applied beforehand to assist in adhering the false lash to the skin. In one mode of operation, a user can simply clean or rinse the lash base after use with water so that the lashes can be reused. Unlike the other false eyelashes, the nano glue is already built-in on the lashes. There is no need to apply a layer of glue on the eyelash or eyeliner on eyelid before putting on the lashes.

In a preferred embodiment, the disclosed system includes: (1) a false eyelash with a base; (2) a pressure sensitive adhesive that has preferably been pre-applied to the base of the false eyelash; and (3) an eyeliner with nano technology that increases the adherence of the nano glue an eyelid of the user. In one embodiment, an ingredient of the cooperating eyeliner and nano glue is polyvinylpyrrolidone.

In one mode of use, the eyeliner may be applied to an eyelid in the manner known to those of skill in the art of makeup application. Next, a false eyelash with pre applied nano glue may be adhered to the eye via contacting the nano glue and the eyeliner. After use, the lash may be rinsed with water and set aside for another application to the eyelid of the user while the eyeliner may be removed in the typical manner known to those of skill in the art of makeup removal.

### BRIEF DESCRIPTION OF THE SEVERAL VIEWS OF THE DRAWINGS

Other objectives of the disclosure will become apparent to those skilled in the art once the invention has been shown and described. The manner in which these objectives and other desirable characteristics can be obtained is explained in the following description and attached figures in which:

FIG. 1 is an exemplary drawing of false eyelashes 1000;

FIG. 2 is a cross-section of the false eyelashes 1000 of FIG. 1; and,

FIG. 3 is a diagram for applying the false eyelashes 1000 to the eyelid of a user's eye 2000;

FIG. 4 is a flow chart for preparing an eyeliner.

It is to be noted, however, that the appended figures illustrate only typical embodiments of this invention and are therefore not to be considered limiting of its scope, for the invention may admit to other equally effective embodiments that will be appreciated by those reasonably skilled in the relevant arts. Also, figures are not necessarily made to scale but are representative.

### DETAILED DESCRIPTION OF PREFFERED EMBODIMENTS

Disclosed is a system of false eyelashes that includes: (1) a false eyelash with a base; (2) a pressure sensitive adhesive that has preferably been pre-applied to the base of the false eyelash; and (3) an eyeliner with a formulation that increases the adherence of the pressure sensitive adhesive to the an eyelid of the user. In use, the eyeliner cooperates with the pressure sensitive adhesive to increase the adhesion of the false eyelashes to a user's eyelash and to extend the life of the pressure sensitive adhesive. The more specific details of the disclosed system are described with reference to the attached figures.

False Eyelashes

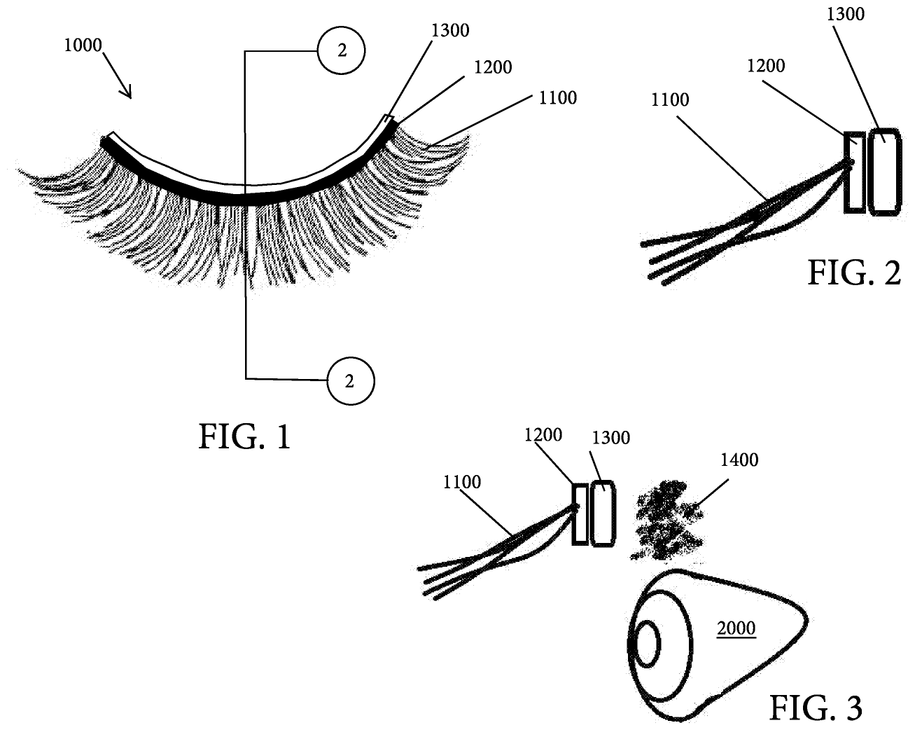

FIG. 1 is an exemplary drawing of false eyelashes 1000. FIG. 2 is a cross-section of the false eyelashes 1000 of FIG. 1. As shown, the false eyelashes 1000 comprise: a base 1200 of string or ribbon like material; hair-like fibers or strands 1100 tied or adhered to the base 1200 so that the fibers or strands 1000 extend from one side of the base 1200; and, a pressure sensitive adhesive 1300 that has been applied to the opposite side of the base 1200 relative to the fibers or strands 1100. In one embodiment, the hair-like fibers or strands are animal hair (e.g., mink hair) and the base 1200 is a thread or ribbon flexible material (e.g., silk, nylon, or other plastic material). The details of the pressure sensitive adhesive are disclosed more fully below.

FIG. 3 is a diagram for applying the false eyelashes 1000 to the eyelid of a user's eye 2000. As can be appreciated by those of skill in the art of installing false eyelashes on the eyelid of a user, an application of eyeliner 1400 that has been formulated to cooperate with the pressure sensitive adhesive 1300 may be applied to the eyelid of a user above the user's eyeball 2000. Next, the pressure sensitive adhesive 1300 of the false eyelashes 1000 may be pressed against the applied eyeliner 1400. As can be appreciated by those of skill in the art of false eyelashes installation, the lash may be secured to the eyelid of a user above the user's natural eyelashes (not shown). After use, the false lashes 1000 may be disconnected from the eyeliner 1400, the false lashes 1000 rinsed with water so that there is no residue of the eyeliner 1400 on the pressure sensitive adhesive 1300, and the false lashes may be set aside for another use in the manner described above with reference to FIG. 3.

Pressure Sensitive Adhesive

In a preferred embodiment, the pressure sensitive adhesive (1300, FIGS. 1-3) is derived by mixing two formulas according to the below specified process. The first formula is a substance that has the appearance of a creamy white gel, a pH of approx 7.0, a viscosity of approx. 8000-30000 mpa·s, a solid content of 50 plus 1 WT %. Suitably, the ingredients are:

an acrylic polymer (approx. 50+1 WT %), an
emulsifier (approx. 1 WT %), and
deionized water (approx. 50 WT %).
Suitably, cyanoacrylate may be the acrylic polymer and Glycerin may be the emulsifier. The second formula is a substance that is a milky white liquid with a mild odor, solid contents (%) of approx. 50 to 52, a pH of approx. 6 to 8, a boiling point greater than 100° C., and a specific gravity at 25° C. of 1.5 plus or minus 0.08. The ingredients of the second formula are:
water (45.6 WT %) (CAS #7732-18-5),
acrylic acid (2.8 WT %) (CAS #79-10-7), and
2-ethylhexyl acrylate (51.6 WT %) (CAS #103-11-7).
The resulting pressure sensitive adhesive is soluble in water. Suitably, the pressure sensitive adhesive is produced by: (1) mixing the ingredients of the first formula (including the acrylic polymer, emulsifier, and deionized water) and then stirring the mixture at 45-50° C. for an hour; (2) letting the mixture cool to 40° C.; (3) adding the ingredients from the second formula; and, (4) stirring the new mixture at 45-50° C. for an hour to obtain the pressure sensitive adhesive. Suitably, the ratio of the first formula to the second formula is 1:1.

In an alternative embodiment, the pressure sensitive adhesive is prepared by emulsion polymerization. Suitably, pressure sensitive adhesive has the following six ingredients: Polyvinylpyrrolidone (cross-lined) (CAS #25249-54-1); polyvinyl alcohol (CAS #9002-89-5); Glycerin (CAS #56-81-5), pure water (CAS #7732-18-5), hydrochloric acid (CAS #111-30-8), and glutaraldehyde (CAS #111-30-8). Suitably, the pressure sensitive adhesive is produced by:

(1) reflux stirring the polyvinylpyrrolidone, polyvinyl alcohol, glycerin, and water at 75-85° C. for 0.5 to 1.5 hours;
(2) letting the mixture cool to 40-50° C.;
(3) adding the Glutaraldehyde; and,
(4) stirring the new mixture at 45-50° C. while there is a reaction for approx. 0.3 to 1 hour to obtain the pressure sensitive adhesive.
Other alternative pressure sensitive adhesives are disclosed by CN107629735A (pub. Jan. 26, 2018) and this document is hereby incorporated by reference in its entirety.

The Cooperating Eyeliner

As set forth above in connection with FIG. 1 through 3, an eyeliner 1400 can be used to increase the adhesions of the eyelashes to the eyelid of a user. In one embodiment, the disclosed eyeliner has the following ingredients:

1) water (CAS #7732-18-5),

### 2) Polyvinylpyrrolidone (CAS #9003-39-8),

3) hydroxyethylcellulose (CAS #9004-62-0),
4) styrene/acrylates copolymer (CAS #9010-92-8),
5) propylene glycol (CAS #57-55-6),
6) polyglyceryl-3 isostearate (CAS #127512-63-4),
7) hydrogenated castor oil (CAS #8001-78-3),
8) ferric oxide (CAS #1309-37-1),
9) tocopherol/Vitamin E (CAS #10191-41-0), and

### 10) Caprylyl Glycol/1,2-Octanediol (CAS #1117-86-8).

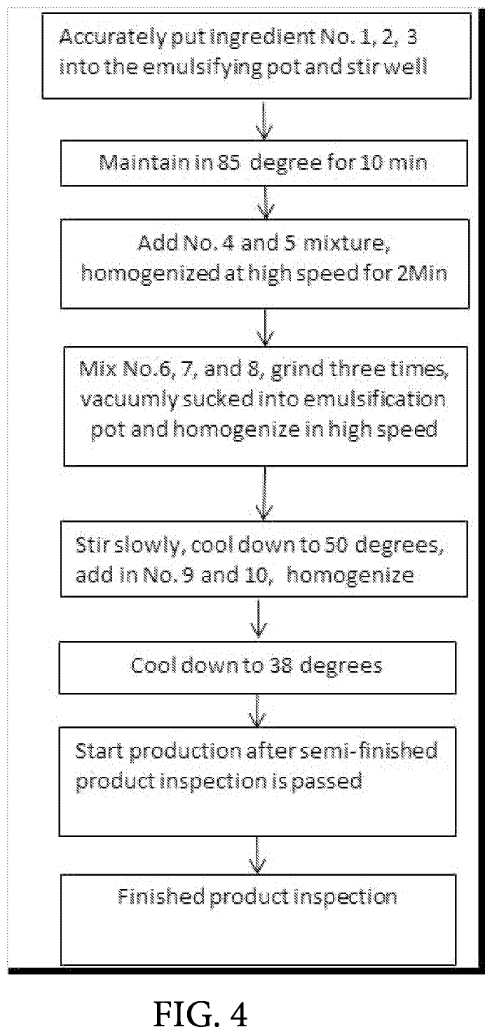

FIG. 4 is a flowchart for preparing the eyeliner. As shown in FIG. 4, the first step is to accurately weigh and put ingredients 1), 2) and 3) into an emulsifying pot. The second step is heating the pot to 85 degrees and maintaining the heat for 10 minutes. The third step is to add ingredients 4) and 5) to the mixture and homogenize at high speed for two minutes. The fourth step is to accurately weigh and mix ingredients 6), 7), and 8) into the emulsifying pot, grind three times, vacuum suck into the emulsification pot and homogenize in high speed. The fifth step is to stir the mixture slowly while the mixture is cooled to 50 degrees before adding ingredients 9) and 10) to the mixture until it is homogenized. The sixth step is to cool the mixture to 38 degrees and then discharge the mixture. The seventh step is to start production after inspection has passed (i.e., after the inspection of the semi-finished product has passed, confirm that the machine, environmental hygiene, packaging and personal hygiene are qualified, then do the filing packaging). The eight step is the finished product inspection and outgoing product shipment.

In another preferred embodiment, the disclosed eyeliner has the following ingredients:

a) water (CAS #7732-18-5) (content 49.5%),
b) Polyvinylpyrrolidone (CAS #9003-39-8) (content 2.0%),
c) hydroxyethylcellulose (CAS #9004-62-0) (content 1.0%),
d) styrene/acrylates copolymer (CAS #9010-92-8) (content 30.0%),
e) propylene glycol (CAS #57-55-6) (content 5.0%),
f) Sodium Benzoate (CAS #532-32-1) (content 1.0%),
g) Propylparaben (CAS #94-13-3) (content 4.5%),
h) ferric oxide (CAS #1309-37-1) (content 6.0%),
i) tocopherol/Vitamin E (CAS #10191-41-0) (content 0.5%), and
j) Caprylyl Glycol/1,2-Octanediol (CAS #1117-86-8) (content 0.5%).

FIG. 4 is a flowchart for preparing the eyeliner. As shown in FIG. 4, the first step is to accurately weigh and put ingredients a), b) and c) into an emulsifying pot. The second step is heating the pot to 85 degrees and maintaining the heat for 10 minutes. The third step is to add ingredients d) and e) to the mixture and homogenize at high speed for two minutes. The fourth step is to accurately weigh and mix ingredients f), g), and h) into the emulsifying pot, grind three times, vacuum suck into the emulsification pot and homogenize in high speed. The fifth step is to stir the mixture slowly while the mixture is cooled to 50 degrees before adding ingredients i) and j) to the mixture until it is homogenized. The sixth step is to cool the mixture to 38 degrees and then discharge the mixture. The seventh step is to start production after inspection has passed (i.e., after the inspection of the semi-finished product has passed, confirm that the machine, environmental hygiene, packaging and personal hygiene are qualified, then do the filing packaging). The eight step is the finished product inspection and outgoing product shipment.

The liner material has certain physical and chemical properties. The liner material may be a black or clear odorless liquid with a flash point of 93 degrees Celsius. The liner material is water soluble and chemically stable. The liner is incompatible with strong oxidizing agents. The liner material should be kept out of direct sunlight and away from high temperatures and open fires. The liner produces no hazardous decomposition products under normal use and storage conditions.

The liner material has relevant toxicological considerations. The product ingredients are considered safe for cosmetics and personal care products. The liner is nonirritant to the skin of people who are not allergic to the liners ingredients however the liner may be an irritant to the eyes. The liner is not known to cause respiratory or skin sensitization.

The liner material also has environmental considerations. The liner is not comprised of hazardous or toxic substances and has a negligible environmental impact based on the liner's use pattern. Further the liner is naturally biodegradable. The potential for bioaccumulation of the liner material in aquatic organisms is low. The liner material may penetrate soil and may be dissolved in the soil material. Under environmental conditions, the liner material is not expected to have a detrimental effect on plants, animals or microorganisms.

One must also make special considerations when disposing of the liner material. Before disposing of the liner material, one may refer to the relevant national and local laws and regulation. Generally, the generation of waste should be avoided or minimized wherever possible. One may dissolve or mix the liner material with a combustible solvent and burn it in a chemical incinerator equipped with an afterburner and scrubber system.

The liner of this preferred embodiment is not hazardous. It may suitably be considered a non-hazardous substance or mixture under the GHS hazard class standard and should therefore may not have GHS label elements or hazard statements. That said, ingesting the liner could, in some cases, cause stomachache. Additionally, if the liner makes eye contact with a user, this contact could potentially cause redness pain and or tears. Although the liner may cause eye pain, the liner does not have any environmental hazards.

First aid measures related to the liner may be necessary. Measures are likely to be necessary if liner material contacts skin, eyes, is ingested, or is inhaled. It is generally advised that an exposed person or their caretaker show a doctor in attendance a document containing liner information such as a material safety data sheet. After receiving the first-aid measure required, the exposed person may continue to consult the doctor if necessary.

If the liner contacts skin or clothes, the exposed person may start by removing contaminated clothing and shoes. Then the exposed person may wash off the contaminated area with mild soap and water. And, if skin irritation occurs or persists, the exposed person may consult a physician.

If material contacts eyes, the exposed person may check for and remove any contact lenses, occasionally lifting the upper and lower eyelids. Then it is recommended that the exposed person immediately flush their eyes with running water. It is also worth noting that the exposed person should not hand rub their eyes.

If material is inhaled, then one may move the exposed person to fresh air and help the exposed person maintain an open airway. It is also considered best practice to keep the exposed person warm and at rest. And, the exposed person may seek medical attention if adverse health effects persist or are severe.

If a person ingests liner the exposed person may wash their mouth out with water. Ideally, the exposed person will be moved to fresh air and kept warm and at rest. If the liner material has been swallowed and the exposed person is conscious, one may give the exposed person small quantities of water to drink. However, stop giving the exposed person water if the exposed person feels sick. Vomiting may be dangerous and should not be induced unless directed by medical personnel. If vomiting occurs, the head of the exposed person should be kept low so that vomit does not enter the lungs.

The liner material exhibits certain characteristics when ignited. If the liner material is ignited, carbon oxides, nitrogen oxides, metal oxides may be released. Ignited liner material may be extinguished with dry sand, dry chemical or CO₂ foam but not a solid water stream.

In the event of a liner material spill the following personal precautions, protective equipment and emergency procedures may be adhered to. Safety is a primary priority. It is recommended that no action is taken involving any personal risk or without suitable training. Further, surrounding areas may be evacuated and unnecessary and unprotected personnel may be kept from entering surrounding areas. It may be necessary to not touch or walk through spilt material, in order to avoid slipping. If steam is emanating from the spill avoid breathing the steam and if possible, provide adequate ventilation to the area. If it is not possible to provide adequate ventilation, then it is recommended that those who may be exposed wear a respirator and other appropriate personal protective equipment.

It may also be prudent to safely prevent further leakage or spillage in order to avoid environmental discharge. In all events safety is paramount and leaks may be stopped if there is little or no associated risk. If the spill is small, move containers from a spill area then wash the leakage with water. If the spill is large, start by moving a plurality of containers from spill area. Then approach the release from upwind. If possible, prevent entry of the material into sewers, water courses, basements or confined areas. If possible, wash spillages into an effluent treatment plant. One may contain and collect spillage with non-combustible, absorbent material such as sand, earth, vermiculite or diatomaceous earth. However, contaminated absorbent material may pose the same hazard as the spilt product. Thus, adhered or collected material should be promptly disposed of in accordance with appropriate laws and regulations.

There are precautions for safe handling and storage associated with the liner material. Precautions for safe handling involve wearing appropriate personal protective equipment. Further, eating, drinking and smoking should be prohibited in areas where this material is handled, stored, and processed. Prior to eating, drinking, or smoking, workers should wash their hands and face. Workers should also avoid contacting eyes with, and breathing steam from, the material. Work areas should be outfitted with exhaust equipment and be well ventilated. Normal fire prevention measures should be in place, such as control of heat, sparks, open flames, and hot surfaces. Basic housekeeping procedures and hygiene practices should be observed by all personnel. Containers and their packaging should be handled carefully to mitigate damage to the packaging and container. After handling packaging and containers may be wash thoroughly. Further, people with a history of skin sensitization problems should not be employed in any process in which this product is used.

The liner material also has storage precautions which should be adhered to. Liner material may be stored in a dry, cool and well-ventilated area in accordance with local regulations. In storage, containers should be kept tightly closed, away from sparks, open flames, high temperatures, and incompatible materials. Storage areas may be equipped with corresponding varieties and number of fire equipment and spill contingency processing equipment. Lastly, containers may be kept out of reach children and pets.

The liner material has certain personal protective equipment requirements. A multi-function respirator is recommended for respiratory protection if exposure limits are exceeded or if irritation or other symptoms are experienced. Although normally not required if used as intended, eye protection such as safety eyewear complying with an approved standard should be used when a risk assessment indicates eye protection is necessary to avoid exposure to liquid splashes, mists, or dusts. Skin and body protection are normally not required if used as intended. However, wearing protective clothing and boots is recommended during production. Hand protection is recommended during production. Gloves should be checked for their proper condition prior to each use. Glove selection is contingent on the material.

Although the method and apparatus is described above in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting to the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives might be implemented without confinement to the illustrated examples. For example, block diagrams and their accompanying description should not be construed as mandating a particular architecture or configuration.

All original claims submitted with this specification are incorporated by reference in their entirety as if fully set forth herein.

I claim:

1. A system of false eyelashes comprising:
a. At least one prosthetic eyelash defined by a base, and at least one hair-like material secured to the base;
b. A pressure sensitive adhesive applied to the base, wherein the pressure sensitive adhesive was made by:
i. stirring an acrylic polymer (approx. 50+1 WT %), an emulsifier (approx. 1 WT %) and deionized water (approx. 50 WT %) at 45-50° C. for an hour to accomplish a mixture; and,
ii. letting the mixture cool to 40° C.;
c. Eyeliner applied to an eyelid, wherein the eyeliner has the following ingredients:
i. water (CAS #7732-18-5),
ii. Polyvinylpyrrolidone (CAS #9003-39-8),
iii. styrene/acrylates copolymer (CAS #9010-92-8),
iv. propylene glycol (CAS #57-55-6),
v. polyglyceryl-3 isostearate (CAS #127512-63-4),
vi. hydrogenated castor oil (CAS #8001-78-3),
vii. ferric oxide (CAS #1309-37-1),

2. The system of false eyelashes of claim 1 wherein the eyeliner further comprises Caprylyl Glycol/1,2-Octanediol (CAS #1117-86-8).

3. The system of false eyelashes of claim 2 wherein the eyeliner further comprises Tocopherol/Vitamin E (CAS #10191-41-0).

4. The system of false eyelashes of claim 3 wherein the eyeliner further comprises Hydroxyethylcellulose (CAS #9004-62-0).

5. The system of false eyelashes of claim 4 wherein the pressure sensitive adhesive is further made by adding to the mixture water (45.6 WT %) (CAS #7732-18-5), acrylic acid (2.8 WT %) (CAS #79-10-7), and 2-ethylhexyl acrylate (51.6 WT %) (CAS #103-11-7) to accomplish a second mixture.

6. The system of false eyelashes of claim 5 wherein the pressure sensitive adhesive is further made by stirring the second mixture at 45-50° C. for an hour to obtain the pressure sensitive adhesive;

7. The system of false eyelashes of claim 6 wherein the eyeliner further comprises hydrogenated castor oil (CAS #8001-78-3).

8. A system of false eyelashes comprising:
a. At least one prosthetic eyelash defined by a base, and at least one hair-like material secured to the base;
b. A pressure sensitive adhesive applied to the base, wherein the pressure sensitive adhesive was made by:
i. stirring an acrylic polymer (approx. 50+1 WT %), an emulsifier (approx. 1 WT %) and deionized water (approx. 50 WT %) at 45-50° C. for an hour to accomplish a mixture;
ii. letting the mixture cool to 40° C.;
iii. adding to the mixture water (45.6 WT %) (CAS #7732-18-5), acrylic acid (2.8 WT %) (CAS #79-10-7), and 2-ethylhexyl acrylate (51.6 WT %)(CAS #103-11-7) to accomplish a second mixture;
iv. stirring the second mixture at 45-50° C. for an hour to obtain the pressure sensitive adhesive
c. Eyeliner applied to an eyelid, wherein the eyeliner has the following ingredients:
i. water (CAS #7732-18-5),
ii. Polyvinylpyrrolidone (CAS #9003-39-8),
iii. hydroxyethylcellulose (CAS #9004-62-0),
iv. styrene/acrylates copolymer (CAS #9010-92-8),
v. propylene glycol (CAS #57-55-6),
vi. Sodium Benzoate (CAS #532-32-1),
vii. Propylparaben (CAS #94-13-3),
viii. ferric oxide (CAS #1309-37-1),
ix. tocopherol/Vitamin E (CAS #10191-41-0), and
x. Caprylyl Glycol/1,2-Octanediol (CAS #1117-86-8)

9. The system of false eyelashes of claim 8 wherein the eyeliner ingredient Polyvinylpyrrolidone (CAS #9003-39-8) has a 2.0% content.

10. The system of false eyelashes of claim 9 wherein the eyeliner ingredient hydroxyethylcellulose (CAS #9004-62-0) has a 1.0% content.

11. The system of false eyelashes of claim 10 wherein the eyeliner ingredient styrene/acrylates copolymer (CAS #9010-92-8) has a 30.0% content.

12. The system of false eyelashes of claim 11 wherein the eyeliner ingredient propylene glycol (CAS #57-55-6) has a 5.0% content.

13. The system of false eyelashes of claim 12 wherein the eyeliner ingredient Sodium Benzoate (CAS #532-32-1) has a 1.0% content.

14. The system of false eyelashes of claim 13 wherein the eyeliner ingredient Propylparaben (CAS #94-13-3) has a 4.5% content.

15. A method of producing an eyeliner comprising:
Weighing and put water (CAS #7732-18-5) (content 49.5%), Polyvinylpyrrolidone (CAS #9003-39-8) (content 2.0%), hydroxyethylcellulose (CAS #9004-62-0) (content 1.0%), into an emulsifying pot;
heating a pot to 85 degrees;
maintaining the pot at 85 degrees for 10 minutes; and,
adding d) styrene/acrylates copolymer (CAS #9010-92-8) (content 30.0%), e) propylene glycol (CAS #57-55-6) (content 5.0%), to the mixture and homogenize at high speed for two minutes.

16. The method of claim 15 further comprising weighing and mixing ingredients Sodium Benzoate (CAS #532-32-1) (content 1.0%), Propylparaben (CAS #94-13-3) (content 4.5%), ferric oxide (CAS #1309-37-1) (content 6.0%), into the emulsifying pot, grind three times, vacuum suck into the emulsification pot and homogenize in high speed.

17. The method of claim 16 further comprising stirring the mixture slowly while the mixture is cooled to 50 degrees and adding ingredients tocopherol/Vitamin E (CAS #10191-41-0) (content 0.5%), and Caprylyl Glycol/1,2-Octanediol (CAS #1117-86-8) (content 0.5%) to the mixture until it is homogenized.

18. The method of claim 17 further comprising cooling the mixture to 38 degrees and then discharging the mixture.

19. The method of claim 18 further comprising starting production after inspection has passed (i.e., after the inspection of the semi-finished product has passed, confirm that the machine, environmental hygiene, packaging and personal hygiene are qualified, then do the filing packaging).

20. The method of claim 19 further comprising finishing product inspection and product shipment.
